(19) United States
(12) Patent Application Publication
(10) Pub. No.: US 2026/0283672 A1
(43) Pub. Date: Sep. 24, 2026

(54) CATHETER WITH DEFORMATION SENSING

(71) Applicant: Boston Scientific Scimed, Inc., Maple Grove, MN (US)

(72) Inventors: Nathan Paul HAGSTROM, Blaine, MN (US); Michael Sean COE, Plymouth, MN (US)

(21) Appl. No.: 19/574,073

(22) Filed: Mar. 20, 2026

Related U.S. Application Data
(60) Provisional application No. 63/775,781, filed on Mar. 21, 2025.

(51) Int. Cl. A61B 18/00; A61B 90/00

(52) U.S. Cl. CPC A61B 18/00; A61B 90/06; A61B 2018/0016; A61B 2018/00351; A61B 2018/00577; A61B 2018/00613; A61B 2018/00875; A61B 2018/00892; A61B 2018/00904; A61B 2090/064

(57) ABSTRACT

A catheter for ablating cardiac tissue through irreversible electroporation is disclosed. The catheter includes an electrode assembly extending distally from a distal end of an outer shaft. The electrode assembly defines a distally located central hub portion and splines each including a distal end portion extending from the central hub portion, a proximal end portion constrained by the outer shaft, and an intermediate portion disposed between the distal end portion and the proximal end portion. Each of the splines also includes an outwardly facing portion and an inwardly facing portion. The electrode assembly includes an outwardly facing flexible circuit disposed on the outwardly facing portions that includes an outwardly facing ablation electrode. Each of the splines also includes an inwardly facing flexible circuit disposed on the inwardly facing portions that includes an inwardly facing strain gauge on the inwardly facing and intermediate portions of the splines.

### CROSS REFERENCE TO RELATED APPLICATIONS

The present application claims priority to U.S. Provisional Patent Application No. 63/775,781, filed Mar. 21, 2025, the disclosure of which is incorporated herein in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radiofrequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a catheter for ablating cardiac tissue through irreversible electroporation, the catheter comprising: an outer shaft having a proximal end and an opposite distal end; an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising: an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end; and an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines.

In Example 2, the catheter of Example 1, further comprising a plurality of spline sensing electrodes located on each spline.

In Example 3, the catheter of Example 2, wherein the plurality of spline sensing electrodes are disposed on the outwardly facing flexible circuit.

In Example 4, the catheter of any of Examples 1-3, wherein each spline of the plurality of splines includes an inwardly facing flex circuit branch of the plurality of inwardly facing flex circuit branches.

In Example 5, the catheter of any of Examples 1-3, wherein each spline of a subset of the plurality of splines includes an inwardly facing flex circuit branch of the plurality of inwardly facing flex circuit branches.

In Example 6, the catheter of Example 5, wherein the plurality of splines includes six splines and the subset of the plurality of splines includes three splines.

In Example 7, the catheter of any of Examples 4-6, wherein each spline of the plurality of splines includes a respective outwardly facing flex circuit branch of the plurality of outwardly facing flex circuit branches.

In Example 8, the catheter of any of Examples 4-7, wherein each inwardly facing flex circuit branch includes one strain gauge.

In Example 9, the catheter of any of Examples 1-8, further comprising a hub sensing electrode centrally located on the central hub portion of the electrode assembly and a shaft electrode on the outer shaft proximal to the distal end of the outer shaft.

In Example 10, the catheter of any of Examples 1-9, further comprising a post electrode extending distal to the distal end of the outer shaft.

In Example 11, the catheter of any of Examples 1-10, wherein catheter is included in a system for ablating cardiac tissue through irreversible electroporation, the system further comprising: a graphical display; and a controller coupled to the graphical display and the catheter, the controller configured to: determine an impedance from a four-terminal configuration, wherein a current is injected to a current carrying pair of the plurality of electrodes and a volage is measured across a sensing pair of electrodes comprising a plurality of sensing electrodes; determine a force applied to the plurality of splines from sensor strain signals received from the inwardly facing strain gauge; and facilitate a visualization of a representation of the electrode assembly indicating which of the plurality of splines and electrodes are in contact with tissue and the state of the electrode assembly under deformation.

In Example 12, the catheter of Example 11, wherein the controller is configured to cycle through several sets of selected sensing pairs of electrodes during an injection of current.

In Example 13, the catheter of any of Examples 11-12, wherein the controller is configured to receive correlative strain to force data from the catheter.

In Example 14, the catheter of Example 13, wherein controller is configured to determine an axisymmetric strain profile of the electrode assembly from the sensor strain signals.

In Example 15, the catheter of Example 14, wherein the controller is configured to apply the correlative strain to force data loaded to the axisymmetric strain profile and with a determined axial and radial compression on the electrode assembly to determine forces applied to the electrode assembly and a direction of the forces.

In Example 16, a catheter for ablating cardiac tissue through irreversible electroporation, the catheter comprising: an outer shaft having a proximal end and an opposite distal end; an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising: an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end; and an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines.

In Example 17, the catheter of Example 16, further comprising a plurality of spline sensing electrodes located on each spline.

In Example 18, the catheter of Example 17, wherein the plurality of spline sensing electrodes are disposed on the outwardly facing flexible circuit.

In Example 19, the catheter of Example 16, wherein each spline of the plurality of splines includes an inwardly facing flex circuit branch of the plurality of inwardly facing flex circuit branches.

In Example 20, the catheter of Example 19, wherein each spline of the plurality of splines includes a respective outwardly facing flex circuit branch of the plurality of outwardly facing flex circuit branches.

In Example 21, the catheter of Example 19, wherein each inwardly facing flex circuit branch includes one strain gauge.

In Example 22, the catheter of Example 16, wherein each spline of a subset of the plurality of splines includes an inwardly facing flex circuit branch of the plurality of inwardly facing flex circuit branches.

In Example 23, the catheter of Example 22, wherein the plurality of splines includes six splines and the subset of the plurality of splines includes three splines.

In Example 24, the catheter of Example 16, further comprising a hub sensing electrode centrally located on the central hub portion of the electrode assembly and a shaft electrode on the outer shaft proximal to the distal end of the outer shaft.

In Example 25, the catheter of Example 16, further comprising a post electrode extending distal to the distal end of the outer shaft.

In Example 26, a system for ablating cardiac tissue through irreversible electroporation, the system comprising: a catheter for ablating cardiac tissue through irreversible electroporation, the catheter comprising: an outer shaft having a proximal end and an opposite distal end; an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising: an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end; and an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines; a graphical display; and a controller coupled to the graphical display and the catheter, the controller configured to: determine an impedance from a four-terminal configuration, wherein a current is injected to a current carrying pair of the plurality of electrodes and a volage is measured across a sensing pair of electrodes comprising a plurality of sensing electrodes; determine a force applied to the plurality of splines from sensor strain signals received from the inwardly facing strain gauge; and facilitate a visualization of a representation of the electrode assembly indicating which of the plurality of splines and electrodes are in contact with tissue and the state of the electrode assembly under deformation.

In Example 27, the system of Example 26, wherein the controller is configured to cycle through several sets of selected sensing pairs of electrodes during an injection of current.

In Example 28, the system of Example 26, wherein the controller is configured to receive correlative strain to force data from the catheter.

In Example 29, the system of Example 28, wherein controller is configured to determine an axisymmetric strain profile of the electrode assembly from the sensor strain signals.

In Example 30, the system of Example 29, wherein the controller is configured to apply the correlative strain to force data loaded to the axisymmetric strain profile and with a determined axial and radial compression on the electrode assembly to determine forces applied to the electrode assembly and a direction of the forces.

In Example 31, the system of Example 26, further comprising a plurality of spline sensing electrodes located on each spline, wherein the plurality of spline sensing electrodes are disposed on the outwardly facing flexible circuit.

In Example 32, the system of Example 26, wherein each spline of the plurality of splines includes an inwardly facing flex circuit branch of the plurality of inwardly facing flex circuit branches, wherein each inwardly facing flex circuit branch includes one strain gauge.

In Example 33, a method for ablating cardiac tissue through irreversible electroporation including a catheter having an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end, and an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines, the method comprising: determining an impedance from a four-terminal configuration, wherein a current is injected to a current carrying pair of the plurality of electrodes and a volage is measured across a sensing pair of electrodes comprising a plurality of sensing electrodes; determining a force applied to the plurality of splines from sensor strain signals received from the inwardly facing strain gauge; and facilitating a visualization of a representation of the electrode assembly indicating which of the plurality of splines and electrodes are in contact with tissue and the state of the electrode assembly under deformation.

In Example 34, the method of Example 33, further comprising receiving correlative strain to force data from the catheter and determining an axisymmetric strain profile of the electrode assembly from the sensor strain signals.

In Example 35, the method of Example 34, further comprising applying the correlative strain to force data loaded to the axisymmetric strain profile and with a determined axial and radial compression on the electrode assembly to determine forces applied to the electrode assembly and a direction of the forces.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is a perspective illustration of a distal portion of a splined catheter for use in the electrophysiology system of FIG. 1, in accordance with embodiments of the subject matter of the disclosure.

FIGS. 2B is an end view illustration of the distal portion of the splined catheter of FIG. 2A.

FIG. 2C is a partial plan view an electrode assembly of the splined catheter shown in two-dimensions of outwardly facing portions of the splines, in accordance with embodiments of the subject matter of the disclosure.

FIG. 2D is a partial plan view the electrode assembly of the splined catheter shown in two-dimensions of inwardly facing portions of the splines, in accordance with an embodiment of the subject matter of the disclosure.

FIG. 2E is a partial plan view the electrode assembly of the splined catheter shown in two-dimensions of inwardly facing portions of the splines, in accordance with an embodiment of the subject matter of the disclosure.

FIG. 3A is a schematic cross-sectional view of a spline of FIG. 2D.

FIG. 3B is a schematic side view of the spline of FIG. 2D.

FIG. 3C is a schematic plan view of a strain gauge for use with the splined catheter.

FIG. 4 is a schematic diagram of an embodiment of a controller for use with the example electrophysiology system of FIG. 1.

FIG. 5 is a block diagram illustrating an embodiment of a process of the controller of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

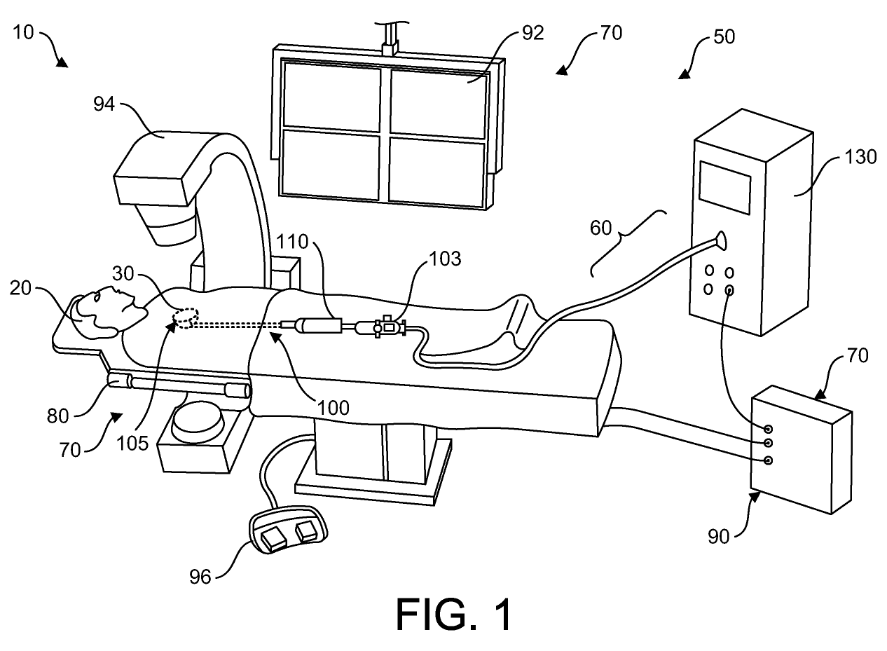

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient’s heart 30. Access to the patient’s heart can be obtained through a vessel, such as a peripheral artery or vein. Once access to the vessel is obtained, the electroporation catheter 100 can be navigated to within the patient’s heart, such as within a chamber of the heart. In embodiments, the electroporation catheter system 60 is configured to deliver electric field energy to targeted tissue in the patient’s heart 30 to create tissue apoptosis, rendering the tissue incapable of conducting electrical signals.

The example electroporation catheter 100 includes an elongated catheter shaft and distal end configured to be deployed proximate target tissue, such as within a chamber of the patient’s heart. The distal end may include a basket, balloon, spline, loop, configured tip, or other electrode deployment mechanism to effect treatment. The electrode deployment mechanism includes an electrode assembly, or array, comprising of an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 100, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or space-apart-time electrical signals, to the electrically connected electroporation catheter 100 along lead conductors to the spaced-apart electrodes. The spaced-apart electrodes are configured to generate a selected electrical field proximate the target tissue, based on the electrical signals from the electroporation console 130, to effect electroporation.

A selected electrical field can be generated with the electrodes to effect electroporation. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 100. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 100 can be operated in a selected mode such as monopolar mode or bipolar mode. During monopolar operation of the catheter 100, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 100, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient’s body, but rather through a localized portion of tissue proximate the electrode assembly.

In some embodiments, the catheter 100 includes a memory device (non-transitory memory) 103 storing a set of parameters associated with the design and configuration of the catheter 100. In one embodiment, the memory device 103 is disposed within the catheter 100, such as within the proximal portion 102 as in a plug or the handle of the catheter 100. The memory device 103 is electrically coupled to the electroporation console 130 via the plug, such as a serial interface, and the electrophysiology system 50 is configured to read the parameters to program the controls to be suited for the associated catheter 100. The memory device 103 can store the parameters in various memory segments having lookup tables or other data structures to provide data to be loaded into a memory device in the electrophysiology system 50, such as a controller of the electroporation console 130 or the EAM system 70, and read by the controller to affect operation. Example parameters can include model number, acceptable voltages or power levels for use with the ablation procedures, whether the catheter 100 is configured for single use or multiple uses, as well as other parameters. In some embodiments, the electrophysiology system 50 can be programmed to write to memory segments on the memory device 103 as well as to read the memory device 103.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the OPAL™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned OPAL HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

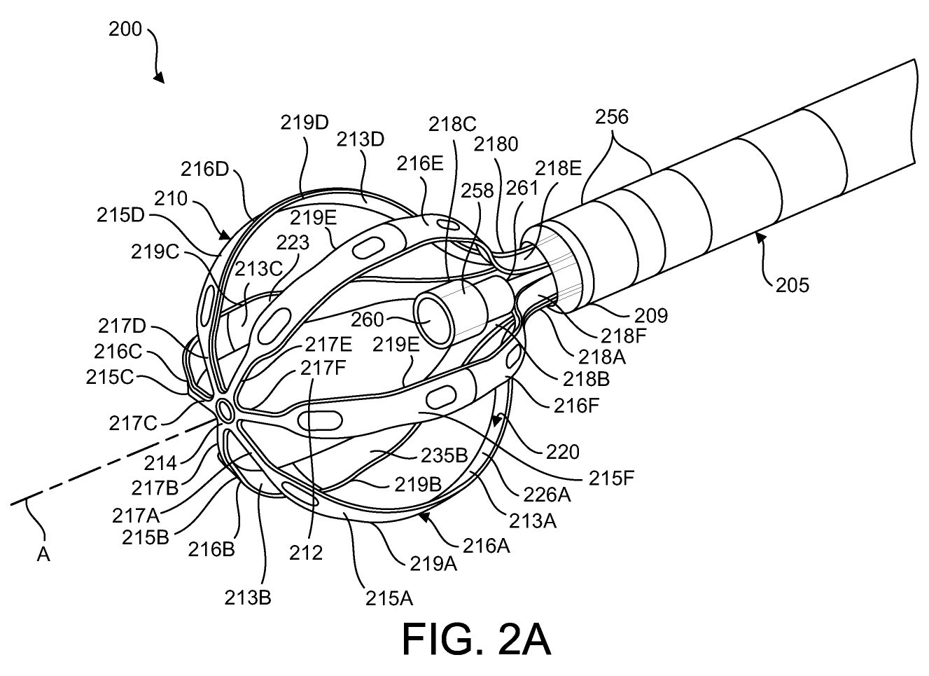

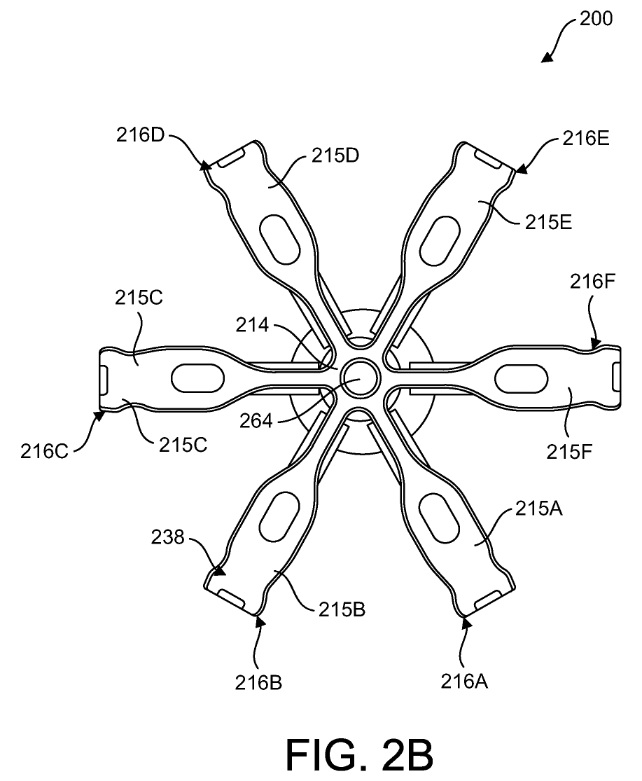

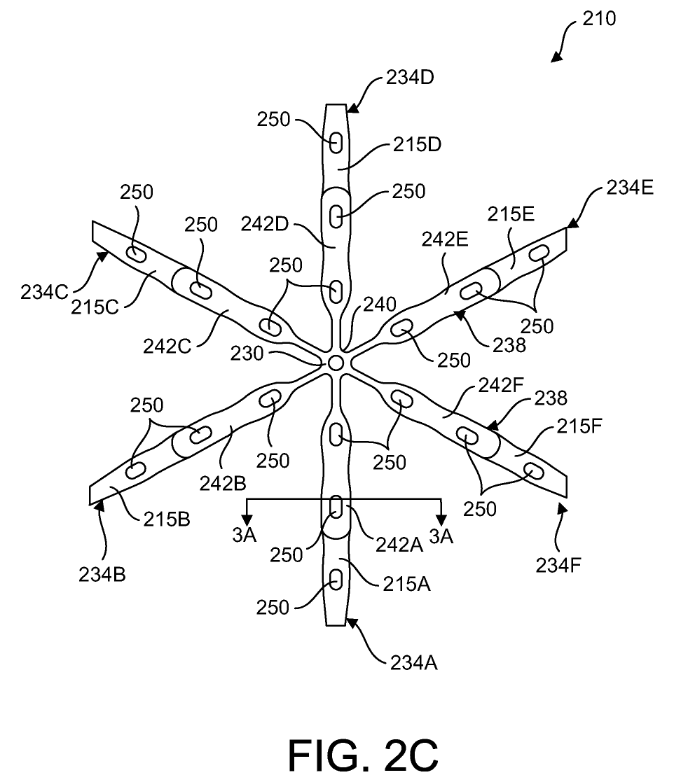

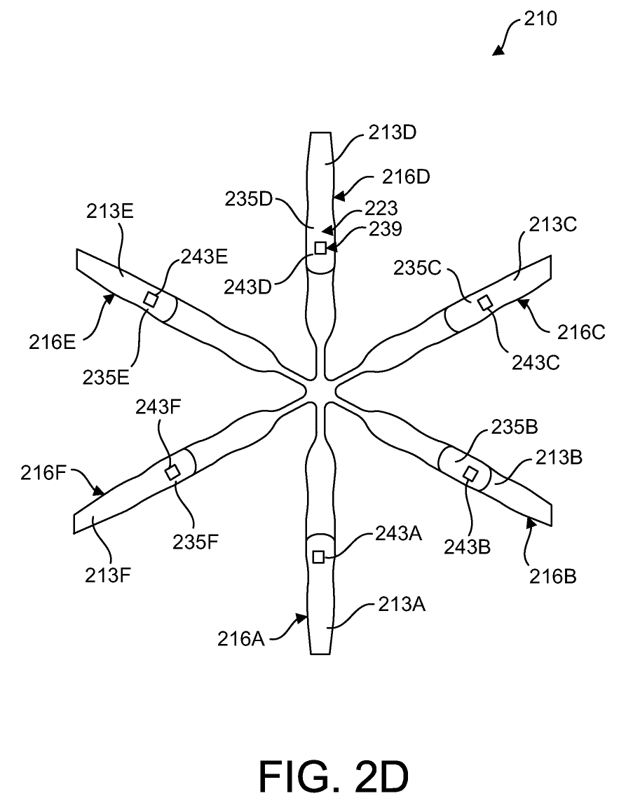

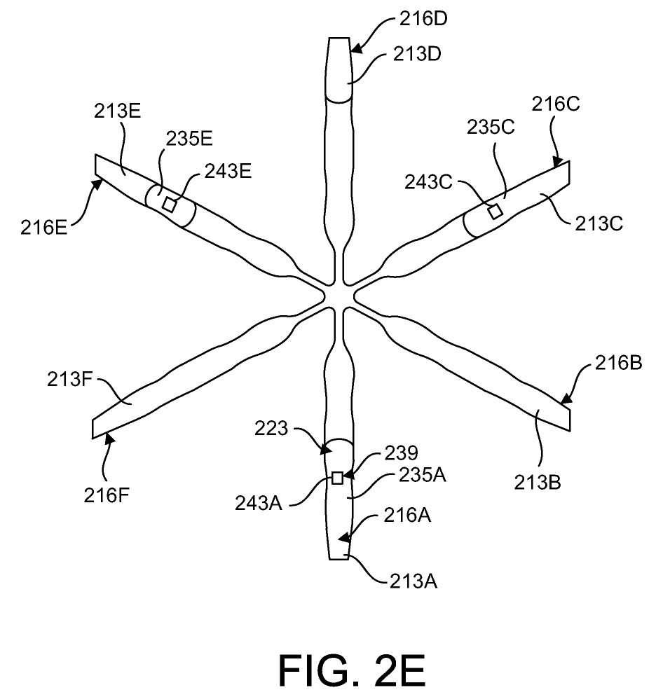

FIGS. 2A and 2B are partial perspective and end view illustrations, respectively, of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 202 having a shaft distal end 209, and an electrode assembly 210 extending distally from the distal end 209 of the outer shaft 202. In embodiments, the electrode assembly 210 is configured to self-expand from a collapsed configuration when constrained within a delivery sheath to a pre-defined expanded configuration defining an inner space 212. As will be explained in greater detail herein, the electrode assembly 210 comprises an ablation electrode configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, is primarily designed for the creation of relatively localized ablation lesions (i.e., focal lesions), as compared to relatively large diameter circumferential lesions created in pulmonary vein isolation procedures. However, the skilled artisan will appreciate that the teachings of the present disclosure can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

The application of flexible circuits to self-expanding catheters enables high-density, high-fidelity electro-anatomical mapping and ablation. Robust and stable catheter to tissue contact is helpful in creating efficacious lesions when using pulse waveforms. Embodiments of the catheter 200 implement electromechanical sensing elements as well as electrical parameter information from electrodes.

In the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective distal end portions 217A-217F. As further shown, each respective spline 216A-216F has an inwardly facing portion 213A-213F directed toward the inner space 212 and an opposite, outwardly facing portion 215A-215F directed away from the inner space 212. In embodiments, the particular geometry of the splines 216A-216F and the related components, e.g., ablation and mapping electrodes, is optimized to provide desired mechanical and therapeutic/diagnostic capabilities.

FIGS. 2C and 2D further illustrate the electrode assembly 210 of the electrode assembly. FIG. 2C is a partial plan view of the electrode assembly 210 of the electroporation catheter 200 shown, shown in two-dimensions to illustrate the layout of the electrode assembly 210 with the outwardly facing portions 215A-215F of the splines 216A-216F in view. FIG. 2D is a partial plan view of the electrode assembly 210 of the electroporation catheter 200 shown, shown in two-dimensions to illustrate the layout of the electrode assembly 210 with the inwardly facing portions 213A-213F of the splines 216A-216F in view.

Referring to FIGS. 2A-2D together, the splines 216A-216F are composed of a support member 220, an outwardly facing flexible circuit 222 secured to and disposed over an outer surface of the support member 220, and an inwardly facing flexible circuit 223 secured to and disposed over the inner surface of the support member 220 in the illustrated embodiments. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy.

The support member 220 includes a support member hub 224 and a plurality of support member branches (for ease of illustration, only support member branch 226A is labeled in FIG. 2A). In embodiments, the support member branches are integrally formed with and extend proximally from the support member hub 224. For example, the entire support member 220 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape such as shown in FIG. 2A due to the shape memory properties of the material, while providing sufficient flexibility necessary to collapse the electrode assembly 210 within a delivery sheath. In embodiments, the support member branches can be selectively configured along their lengths to tune the mechanical characteristics of the electrode assembly 210.

The outwardly facing flexible circuit 222 includes a flex circuit hub 230 and a plurality of outwardly facing flex circuit branches 234A-234F. In embodiments, the flex circuit hub 230 is disposed over and secured to the support member hub 224. In embodiments, the outwardly facing flex circuit branches 234A-234F are integrally formed with the flex circuit hub 230, and each of the outwardly facing flex circuit branches 234A-234F is disposed over and secured to a respective one of the support member branches, such as on an outwardly facing portion. The outwardly facing flexible circuit 222 comprises a layered construction including one or more dielectric substrate layers, and conductive traces formed thereon. The dielectric layers serve to electrically insulate electrodes and traces from each other and the splines. In some embodiments, the flex circuits extend along the shaft, such as along a portion of the shaft or along the entire length of the shaft to the proximal end. Similar to the support member 220, the unitary construction of the outwardly facing flexible circuit 222 enhances its structural properties, for example, by minimizing joints or other discontinuities at regions subject to relatively high stresses during use.

As shown, the outwardly facing flexible circuit 222 includes an outwardly facing ablation electrode 238, or ablation electrode 238, that has an ablation electrode hub portion 240 and a plurality of ablation electrode branches 242A-242F. In the illustrated embodiment, the distal ablation electrode hub portion 240 is located on the flex circuit hub 230. Additionally, the ablation electrode branches 242A-242F are integrally formed with the ablation electrode hub portion 240. Each of the ablation electrode branches 242A-242F extends proximally along a portion of a respective one of the outwardly facing flex circuit branches 234A-234F.

As further shown, the outwardly facing flexible circuit 222 includes a plurality of spline sensing electrodes 250. In the illustrated embodiment, two of the spline sensing electrodes 250 are disposed within a periphery of each of the ablation electrode branches 242A-242F, and one of the spline sensing electrodes 250 is located proximal to each of the ablation electrode branches 242A-242F on a respective outwardly facing flex circuit branch 234A-234F. The illustrated configuration is exemplary only, and other embodiments of the catheter 200 may have alternative configurations. Thus, in various embodiments, one or more of the spline sensing electrodes 250 may be disposed within the periphery of one or more of the ablation electrode branches 242A-242F and electrically isolated therefrom, and one or more of the spline sensing electrodes 250 may be located proximal to the ablation electrode branches 242A-242F on the respective outwardly facing flex circuit branch 234A-234F. In still other embodiments, no spline sensing electrodes 250 may be located outside the peripheries of the ablation electrode branches 242A-242F. The electrodes such as the ablation electrode 238 and spline sensing electrodes 250 are electrically coupled to the conductive traces are electrically coupled to and to conductive leads disposed in the shaft to electrically couple the electrodes to the shaft.

The inwardly facing flexible circuit 223 includes a plurality of inwardly facing flex circuit branches, such as 235A-235F on the inwardly facing portion 213A-213F of at least some of the splines 216A-216F. In one embodiment, each of the splines 216A-216F includes an inwardly facing flex circuit branch 235A-235F. In some embodiments, a subset of the splines, such as three of six splines of the electrode assembly 210 includes an inwardly facing flex circuit branch, such as splines 216A, 216C, 216E include inwardly facing flex circuit branches. The subset of splines is less than all of the splines of the electrode assembly. In embodiments, each of the inwardly facing flex circuit branches 235A-235F is disposed over and secured to a respective one of the support member branches, such as on an inwardly facing portion 213A-213F. The inwardly facing flexible circuit 223 comprises a layered construction including one or more dielectric substrate layers, and conductive traces formed thereon. The dielectric layers serve to electrically insulate electrical components and traces from each other and the splines. In some embodiments, the flex circuits extend along the shaft, such as along a portion of the shaft or along the entire length of the shaft to the proximal end. Similar to the support member 220, the unitary construction of the inwardly facing flexible circuit 223 enhances its structural properties, for example, by minimizing joints or other discontinuities at regions subject to relatively high stresses during use.

As shown, the inwardly facing flexible circuit 223 includes an inwardly facing contact force sensor assembly 239. Embodiments of a contact force sensor assembly 239 include a strain gauge, a piezoelectrical sensor a force sensing resistor, a load cell, and a magnetic force sensor. In some embodiments, all of inwardly facing flex circuit branches included in the electrode assembly (inwardly facing flex circuit branches on subset of all of the splines to all of the spline) includes a contact force sensor assembly 243A-243F. In some embodiments, each of the inwardly facing flex circuit branches 235A-235F includes a plurality of longitudinally spaced apart contact force sensor assemblies 243A-243F. The traces are electrically coupled to the contact force sensor assemblies 243A-243F and to conductive leads disposed in the shaft to electrically couple the electrodes to the shaft.

FIG. 2D illustrates one embodiment of catheter 200 in which the electrode assembly 210 includes an inwardly facing flex circuit branch 235A-235F on each spline 216A-216F. Each of the inwardly facing flex circuit branches 235A-235F extends distally along a portion of a respective one of the inwardly facing portions 213A-213F of the splines 216A-216F from the distal end 209 of the tubular outer shaft 202. In the some embodiments, each flex circuit branch 235A-235F includes a contact force sensor assembly, such as at least one contact force sensor assembly, 243A-243F. In the illustrated embodiment, each flex circuit branch 235A-235F has one contact force sensor assembly, 243A-243F. In the illustrated embodiment, the contact force assemblies 243A-243F are all longitudinally disposed along the inwardly facing flex circuit branch to be positioned on the respective spline 216A-216F at a section of relatively large radius of the spline when the electrode assembly 210 is in the expanded configuration, such as in the intermediate portions 219A-219F. This section, the section of largest radius in the intermediate portions 219A-219F, is typically subjected to the most bending from outside forces of the various regions of the splines 216A-216F when the catheter is in use.

FIG. 2E illustrates another embodiment of catheter 200 in which like parts include like reference numbers. In the embodiment of FIG. 2E, the electrode assembly 210 include an inwardly facing flex circuit branch 235A, 235C, 235E on only three splines 216A, 216C, 216E of the six spline 216A-216F. Each of the inwardly facing flex circuit branches 235A, 235C, and 235E extends distally along a portion of a respective one of the inwardly facing portions 213A, 213C, 213E of the subset of splines 216A, 216C, 216E from the distal end 209 of the tubular outer shaft 202. In some embodiments, each flex circuit branch 235A, 235C, 235E includes a contact force sensor assembly, such as at least one contact force sensor assembly, 243A, 243A, 243C, 243E. In the illustrated embodiment, each flex circuit branch 235A, 235C, 235E has one contact force sensor assembly, 243A, 243C, 243E. In some embodiments, including a contact force sensor assembly on half of the splines to all of the splines enables axis-symmetric assumptions or interpolations in signals indicative of electro-mechanical force. In the illustrated embodiment, the contact force assemblies 243A, 243C, 243E are all longitudinally disposed along the inwardly facing flex circuit branch to be positioned on the respective spline 216A, 216C, 216E at a section of relatively large radius of the spline when the electrode assembly 210 is in the expanded configuration, such as in the intermediate portions 219A-219F.

In some embodiments, the structural functionality of the support member 220 can be provided by suitably designed flexible circuits 222,223. As such, although the electrode assembly 210 is described in detail as including the support member 220 as a primary structural member, in other embodiments the support member 220 can be omitted in its entirety and the corresponding functionality can be provided by the flexible circuits 222,223. In some such embodiments, the flexible circuits 222, 223 are integrally formed as a single flexible circuit having an outwardly facing section corresponding with flexible circuit 222 and an inwardly facing section corresponding with flexible circuit 223.

In the particular illustrated embodiments, the electroporation catheter 200 includes a pair of shaft electrodes 256 located proximate the distal end 209 of the outer shaft 202, as well as a central post 258 extending distally from the distal end 209 of the outer shaft 202. As shown, the central post 258 extends partially into the inner space 212, and includes a post electrode 260. In some embodiments, the central post 258 includes additional components. For example, in some embodiments, a magnetic navigation sensor (not shown) is partially or wholly disposed within the central post 258. However, in other embodiments such a sensor may be located elsewhere on the electroporation catheter 200 (e.g., within the outer shaft 202). In the illustrated embodiment, the electrode assembly 210 further includes a hub sensing electrode 264 centrally located on the flex circuit hub 230. In embodiments, one or both of the shaft electrodes 256 can be configured to be paired with the ablation electrode 238 to form an anode/cathode ablation electrode pair for generation of an ablative electric field in a bipolar mode. In embodiments, the shaft electrodes 256 may have additional functions, e.g., and without limitation, as additional sensing electrodes for sensing cardiac electrical signals, and for use as localization sensors for impedance tracking of the electrode assembly 210.

The post electrode 260 can provide a number of functional advantages. In one example, the post electrode 260 can operate as a reference for unipolar electrograms, in lieu of reliance on surface ECG patch electrodes as are otherwise known in the art. The location of the post electrode 260 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrode 238 or other sensing electrodes 250, 264 on the electrode assembly 210, thereby providing data usable for, in some examples, determining the shape of the electrode assembly during use (including when deformed by forces applied by cardiac walls), and displaying shape information via the EAM system 70 (FIG. 1).

In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

The electrode assembly 210 is configured to be electrically coupled to a source of ablation energy, such as the electroporation console 130 in FIG. 1, to generate electric fields sufficient to achieve the desired clinical effect, in particular ablation of target tissue through irreversible electroporation. In one embodiment, the electrode assembly 210 is operated in a monopolar mode. In one embodiment, the ablation electrode 238 is configurable as one of a cathode and an anode and a pad dispersive electrode located on the patient, typically on the back, buttocks, or other suitable anatomical location during electroporation. An electrical field is formed between an activated ablation electrode 238 of the electrode assembly 210 and the pad dispersive electrode. In one embodiment, the electrode assembly 210 is operated in a bipolar mode. In one embodiment, the ablation electrode 238 is configurable as one of a cathode and an anode and the at least one other electrode, such as the shaft electrode 256 in many embodiments, is configurable as the other of the anode and the cathode to generate the pulsed electric fields. An electrical field is formed between an activated ablation electrode 238 and the other electrode, such as the shaft electrode 256, of the electrode assembly 210.

The electrode assembly 210 is further configured to receive physiological signals such as for generating electro-anatomical maps such as from sensing electrodes 250 and hub sensing electrode 264. In embodiments, the sensing electrodes 250 and hub sensing electrode 264 have a smaller surface area than the ablation electrode 238, which is well suited to detect physiological signals in the heart for electro-anatomical mapping.

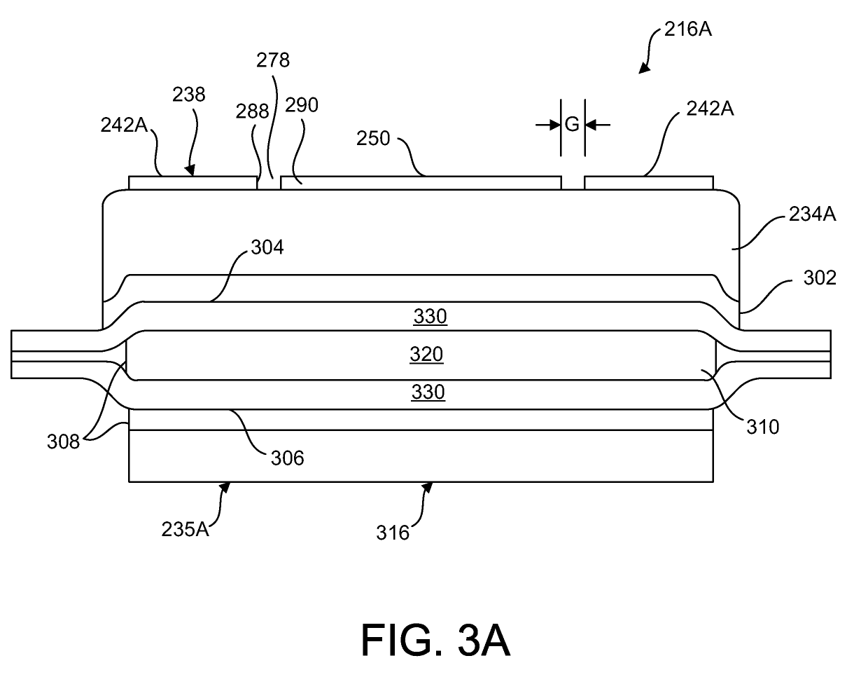

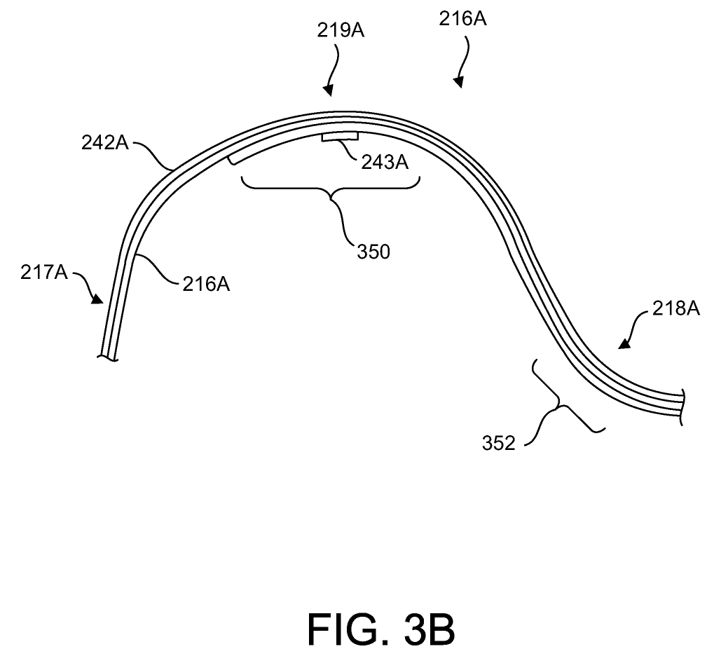

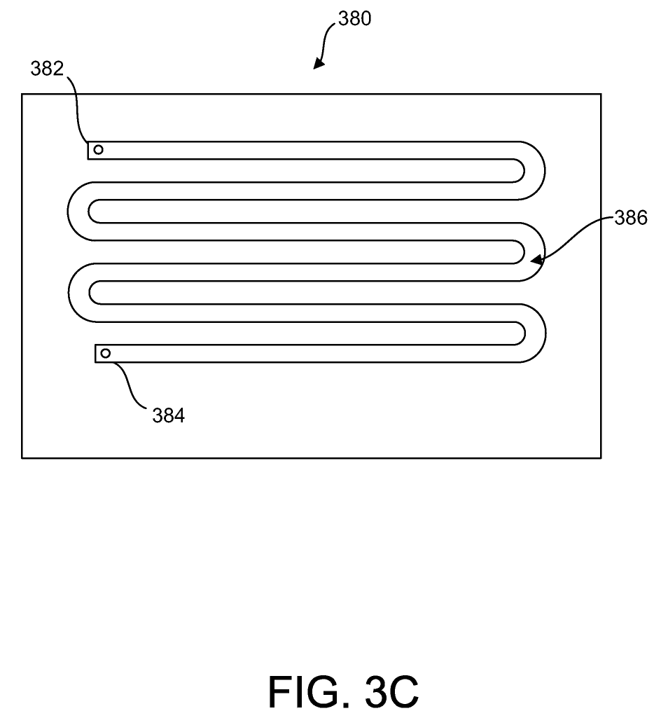

FIG. 3A is a schematic cross-sectional view of the spline 216A taken along the line 3A-3A in FIG. 2C, illustrating an exemplary configuration of the outwardly facing flex circuit branch 234A disposed on the support member branch 226A on the spline 216A. In embodiments, the particular design of the outwardly facing flex circuit branch 234A (and the flex circuit as a whole) can be tailored for the particular clinical needs present. In the particular embodiment illustrated in FIG. 3A, the outwardly facing flex circuit branch 234A is secured to the support member branch 226A by an adhesive layer 302, which may be any suitable adhesive. The support member branch 226A includes an outwardly facing surface 304 and an inwardly facing surface 306. In the illustrated embodiment, the outwardly facing surface 304 and inwardly facing surface 306 are planar, although the surfaces can be curvilinear in other embodiments. In some embodiments, the outwardly facing surface 304 is opposite the inwardly facing surface 306.The outwardly facing flex circuit branch 234A is secured to the outwardly facing surface 304 of the support member branch 226A. The support member branch 226 also includes side surfaces 308, 310.

The outwardly facing ablation electrode 238 and the spline sensing electrode 250 are disposed on an outwardly facing surface 312 of the outwardly facing flex circuit branch 234A. In embodiments, both the outwardly facing ablation electrode 238 and the sensing electrode 250 have a coating of a suitable biocompatible metal, e.g., gold. In embodiments, the outer surfaces of the electrodes may be treated to provide the electrical properties desired for the particular clinical application. The proximal ablation electrode aperture 278 is bounded by an inner peripheral surface 288 of the outwardly facing ablation electrode branch 242A, and an outer peripheral surface 290 of the spline sensing electrode 250 is spaced from the inner peripheral surface 288 of the outwardly facing ablation electrode branch 242A by a gap G. In some embodiments, the gap G and portions of the outwardly facing ablation electrode 238 and the spline sensing electrode 250 may be selectively covered by a dielectric material (not shown).

FIG. 3A also illustrates an exemplary configuration of the inwardly facing flex circuit branch 235A disposed on the support member branch 226A on the spline 216A. In embodiments, the particular design of the inwardly facing flex circuit branch 235A (and the flex circuit as a whole) can be tailored for the particular clinical needs present. In the particular embodiment, the inwardly facing flex circuit branch 235A is secured to the inwardly facing surface 306 of the support member branch 226A by an adhesive layer 312, which may be any suitable adhesive. The force contact assembly 239, not shown in the illustration, is disposed on an inwardly facing surface 316 of the inwardly facing flex circuit branch 235A.

FIG. 3A further illustrates one embodiment of a support member 220 configured to reduce the likelihood of electrically coupling between the support member 220 and the outwardly facing and inwardly facing flex circuit branches 234A, 235A. The support member 220, as illustrated via support member branch 226A, includes an electrically conductive base member 320 covered with an electrically insulative coating 330. In the illustrated example, the electrically insulative coating 330 is a thin film of a dielectric material such as silicone, parylene, polyvinylidene fluoride, or other materials having similar dielectric properties. In one embodiment, the electrically insulative coating 330 is deposited on the base member 320 via an appropriate process including spay coat, dip coat, chemical vapor deposition, and atomic layer deposition, and the like. In one embodiment, the electrically insulative coating 330 encapsulates the entire electrically conductive base member 320 distal to the shaft distal end 209 (see FIG. 2A). The thicknesses of the conductive base member 320 and the coating 330 may be selectively tailored to provide a desired degree of structural support and the aforementioned electrical decoupling. In one exemplary embodiment, the base member 320 may have a thickness of about 68 micrometers, and the dielectric coating 330 may have a thickness of about 12 micrometers, such that the overall thickness of the support member branch 226A is about 92 micrometers.

FIG. 3B illustrates a schematic side view of a portion the spline 216A in the expanded configuration. The schematic side view of the spline 216A illustrates the outwardly facing ablation electrode branch 242A and contact force sensor assembly 243A relative to one another along a longitudinal axis of the catheter 200. In the illustrated embodiment, the proximal end 274A of the outwardly facing ablation electrode branch 242A is distally located along the spline 216A from the contact force sensor assembly 243A. The spline 216A includes a first section 350 of a relatively large radius R1 distal to a second section 352 of a relatively small radius R2 when the electrode assembly 210 is in the expanded configuration. In the illustrated embodiment, the contact force sensor assembly 243A is disposed on the spline 216A to take measurements of force at the first section 350. In some embodiments, a plurality of force sensor assemblies can be disposed on the splines, such as one in the first section 350 and another in the second section. Other configurations are contemplated.

FIG. 3C illustrates a schematic diagram of a plan view of strain gauge 380 for use in a contact force sensor assembly 239 in catheter 200, such as contact force assembly 243A on spline 216A. Each contact force sensor assembly 239 is configured to measure deflection in at least one direction. In some embodiments, the contact force assembly 239 is configured to measure force in a plurality of different directions, such as orthogonal directions, via one or more force sensor elements such as strain gauge 300. The strain gauge 380 is a sensor in which electrical resistance varies with an applied force. The strain gauge 380 converts force, pressure tension, and weight into a change in electrical resistance that can then be measured. In embodiments, the strain gauge is constructed in a manner suitable for use in flexible circuit 223, such as a laminated-type architecture. The strain gauge 380 includes electrical connections 382, 384, that are electrically coupled to the traces to receive an electrical signal, such as a voltage. The strain gauge 380 also includes a deflector portion 386 that changes electrical resistance when a force is applied. The width of the strain gauge can be determined via a width of the spline 216 and the longitudinal length can be selected based on the application or other considerations. Traces in the inwardly facing flex circuit 223 carry the electrical signal across the deflector portion 386, and changes in the electrical characteristics of the electrical signal, such as current or voltage, when the strain gauge is subjected to force can be measured to determine the amount of force or deflection of the spline in the region of the contact force sensor assembly 239. In some embodiments, the strain gauge 380 can be laminated into the flexible circuit 223 or attached to the splines 216A-216F and electrically coupled to the flexible circuit 223.

The electrophysiology system 50 is capable of detecting, or is configured to detect, electrical characteristics, such as impedance, which can correspond with several properties including myocardial tissue proximity to an electrode. For example, the system 50 utilizes impedance measurements to sense contact between an electrode on the catheter 100, such as catheter 200, and tissue. In general, the impedance of a given medium is determined based upon applying a known voltage or current to a given medium and measuring the resulting current or voltage. In some embodiments, impedance measurements of a given medium can be obtained by injecting current between two electrodes and measuring the resulting voltage between the same electrodes through which the current was injected. In one example, the controller, such as controller of the electroporation console 130 or the mapping and navigation controller 90, can select and inject a current between any two electrodes on the catheter 200 and measure a resulting voltage between the same electrodes. In this example, impedance is determined in a two-terminal configuration. The ratio of the voltage potential to the applied current provides an indication of the impedance of the medium through which the current traveled, which can be determined via the controller. For example, a current can be injected between distal ablation electrode 238 and a shaft ring electrode 256. Impedance of the medium (e.g., cardiac tissue) adjacent to distal ablation electrode 238 and shaft ring electrode 256 can be measured according to the methodology disclosed above. For example, if the current is injected between electrodes embedded in cardiac tissue, the impedance of the cardiac tissue may be determined.

In some instances, system 50 may utilize different impedance measurements of a local medium to determine whether the distal ablation electrode 238 is contacting tissue. For example, the impedance of cardiac tissue is different than that of the impedance of blood. Therefore, by knowing the relative difference in the impedances of tissue versus blood, system 50 may be able to determine whether the medium through which a current is being applied is either blood or cardiac tissue, for example.

In some embodiments, impedance can be determined in a four-terminal configuration. In general, the four-terminal configuration drives current through a pair of electrodes of the electrode assembly 210 and measures voltage across a different pair of electrodes. For example, current is injected via a current carrying pair of electrodes and voltage is measured across a sensing pair of electrodes. In one advantageous application, a four-terminal configuration may not be sensitive to the impedance of the electrodes themselves. For instance, the measured impedance in a two-terminal configuration includes the surrounding medium and both electrodes. In contrast, the four-terminal configuration measures the voltage across the sensing pair of electrodes in which the current is negligible. Consequently, the measured impedance is that of the surrounding medium and is largely independent of the impedance of the sensing pair of electrodes and their interface with the surrounding medium.

In one embodiment, a current can be injected between the ablation electrode 238 and a shaft electrode 256, which define the current carrying pair of electrodes. A pair of distal-most spline sensing electrodes 250 on adjacent spline such as splines 216B, 216C can define the sensing electrode pair. The current is injected between the distal ablation electrode 238 and the shaft electrode 256, and voltage is measured across the distal-most spline sensing electrodes 250 on adjacent splines 216B, 216C. The impedance measurement is the impedance of the medium between the sensing pair of electrodes, or the medium adjacent the distal-most spline sensing electrodes 250 on adjacent splines 216B, 216C in this example. For instance, if the distal-most spline sensing electrodes 250 on adjacent splines 216B, 216C are abutted against cardiac tissue, the distal-most spline sensing electrodes 250 on adjacent splines 216B, 216C measure the impedance of the cardiac tissue.

In another embodiment, a current can be injected between the ablation electrode 238 and a shaft electrode 256, and the voltage is measured between a pair of spline sensing electrodes 250, such as a pair of adjacent spline sensing electrode 250, on a spline such as spline 216B. The current carrying pair of electrodes are defined as the ablation electrode 238 and the shaft electrode 256, and the sensing electrode pair are defined as the pair of spline sensing electrodes 250 on the spline such as spline 216C, such as the adjacent pair of spline sensing electrode 250. The impedance measurement is the impedance of the medium between the sensing pair of electrodes, or the medium adjacent the spline sensing electrodes 250 on spline 216B in this example. For instance, if adjacent spline sensing electrodes 250 on spline 216B are abutted against cardiac tissue, the spline sensing electrodes 250 on splines 216B measure the impedance of the cardiac tissue.

In still another embodiment, a current can be injected between the ablation electrode 238 and a shaft electrode 256, and the voltage is measured between the post electrode 260 and a spline sensing electrode 250. In one example, the voltage can be measured between the post electrode and different spline sensing electrodes separately, such as sequentially. For example, a first voltage is measured between the post electrode 260 and a first spline sensing electrode of the spline sensing electrodes, and then a second voltage is measured between the post electrode 260 and a second spline electrode of the spline sensing electrodes 250, and then a third voltage is measured between the post electrode 260 and a third spline electrode of the spline sensing electrodes 250, and so on, until all of the spline sensing electrodes have been employed. In one embodiment, the post electrode 260 is likely not to contact tissue or deviate from position when the splines are deflected, and the measurements can be indicative of deformations in the splines rather than whether a spline is in contact with tissue. For instance, if an impedance measurement deviates or differs from an impedance measurement of the splines in the expanded but undeformed state, the spline electrode employed in the impedance measurement has likely been moved with respect to the post electrode 260. Other current and voltage pairs of electrodes can be applied to determine spline deformation via impedance measurements.

In some embodiments, any pair of electrodes of the electrode assembly 210 can implemented as current carrying pair of electrodes from which current is injected, and any pair of electrodes of the electrode assembly 210 can be implemented as the sensing pair of electrodes across which voltage is measured. In some embodiments, local impedance is measured on each spline 21A-216F using the hub sensing electrode 264 and a spline sensing electrode 250 on one spline as a current carrying pair of electrodes to drive current. In other embodiments, local impedance is measured on each spline 21A-216F using the ablation electrode 238 and one or both of the shaft electrodes 256 as a current carrying pair of electrodes to drive current. In still other embodiments, local impedance can be measured for each spline sensing electrode 250 using the hub sensing electrode 264 and one or both of the shaft electrodes 256 as a current carrying pair of electrodes to drive a current or the hub sensing electrode 264 and the post electrode 260 as a current carrying paid of electrodes to drive a current. In some embodiments. any two sensing electrodes 250, such as sensing electrodes on adjacent splines or adjacent sensing electrodes on the same spline, can comprise a sensing pair of electrodes to measure a voltage. In one embodiment, the two sensing electrodes most proximate to a strain gauge indicating a deflection of the spline can comprise a sensing pair of electrodes to measure voltage. In other embodiments, hub electrode 264 and a spline sensing electrode 250 on a first spline 216A comprise a first sensing pair of electrodes to measure a first volage v 1, two adjacent spline sensing electrodes 250 on the first spline 216A comprise a second sensing pair of electrodes to measure a second voltage v 2, and two other adjacent spline sensing electrodes 250 on the first 216A comprise a third sensing pair of electrodes to measure a third voltage v 3. In one embodiment, the impedance is determined along each spline for each spline in this manner. Different frequencies of the injected current can be applied to determine a preferred frequency for use in the impedance measurement based on different states of cardiac tissue. In one embodiment, impedance measurements between sensing pairs of electrodes spline are multiplexed in time or frequency and measured for each sensing pair of electrodes, and impedance measurements are multiplexed in time or frequency and measured frequently enough to account for movement of the catheter 200.

In some embodiments, the four-terminal configuration is implemented via three electrodes, such as one of the electrodes (a dual-purpose electrode in the configuration) is included in the current carrying pair of electrodes and the sensing pair of electrodes. This configuration can approximate the four-terminal configuration if the impedance of the dual-purpose electrode is low and not expected to vary significantly. Additionally, one or more of the electrodes in a four-terminal configuration can be located off the catheter 200, such as on a pad coupled to the patient’s skin or on another surgical device.

The application of contact force sensing assemblies 239 to generate electro-mechanical strain signals and electrical impedance from the electrodes of the electrode assembly 210 provides advantages. The contact force sensing assemblies 239, such as strain gauges laminated onto the splines 216A-216F, can be applied to determine information regarding deflection of the splines 216A-216F, backing out shape or force applied with shape detection, contact stability, and contact force assessment. Combining the electrical impedance information with the strain data can provide additional information such as magnitude and direction of deformation of the splines, magnitude and direction of force applied to the electrode assembly, and whether or which of the splines are in contact with tissue. Using electrically based features alone to determine contact of the electrode assembly can be difficult. Such techniques are often dependent on electrical properties of the tissue surface, which is modified with pulsed field ablation therapy. For example, tissue treated with pulsed field ablation therapy has a transient conductivity or impedance that makes contact assessment possible but difficult, which reduces confidence levels of contact assessment. Using contact force sensing assemblies alone to determine the shape of the splines of electrode assembly alone also is possible but difficult as multiple shapes can be achieved via a strain state.

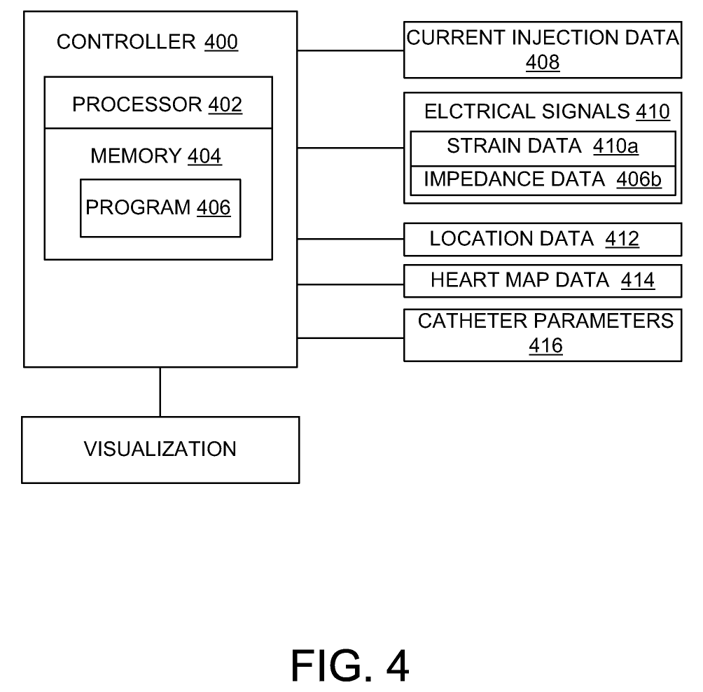

FIG. 4 illustrates an example controller 400 that can be used with the catheter 200 to provide force detection, a high confidence of contact assessment and a high confidence graphical indication of spline deformation. The controller 400 can be implemented with the electrophysiology system 50, such as a controller of the example electroporation catheter system 60, which may include a controller of the electroporation console 130, or a controller of the example EAM system 70, which may include a mapping and navigation controller 90. The controller 400 can include a processor 402 and a memory 404. The memory 404 stores processor executable instructions, such as program 406. In one example, the processor executable instructions 406 can be in the form of a program, such as a computer program or application. In one example, the controller 400 can be implemented to include a computing device such as a laptop computer, a workstation, a desktop computer, or a tablet. The controller 400 can be implemented to include or be coupled to additional components such as a graphical display, a touchscreen, speakers or other output devices, a keyboard or other input devices, or communication circuitry such as computer network adapters. In one example, the controller 400 is coupled to display 92. The controller 400 may be implemented in a variety of architectures and components, such as the processor 402 and memory 404, may be distributed in various locations. In some embodiments, the processor 402 includes one or more main processing cores to run an operating system and perform tasks on an integrated circuit, and the processor 402 can also include built-in logic or a programmable functional unit, also on the same integrated circuit. Memory 404 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 402. Any such computer storage media may be part of the controller 400 and implemented as memory 404. Memory 404 is a non-transitory, processor readable memory device. Accordingly, a propagating signal by itself does not qualify as storage media or memory 404.

The controller 400 is configured to receive inputs to and generate outputs from the electrophysiology system 50. For example, the controller 400 can generate outputs such as current injection data 408 to the console 130 to generate currents in selected electrodes of the catheter 100 to measure impedance. Also, the controller 400 can receive electrical signals 410 from the catheter 200. For example, electrical signals 410 can be received from electrodes on the electrode array in response to signals generated via the catheter, such as injected currents, from strain gauges, pulsed field ablation, or from physiological signals such as EGMs. In one embodiment, electrical signals 410 include electrical signals that are representative of strain data 410a, such as signals from the strain gauges or signal that can converted to strain information, and electrical signals that are representative of impedance data 410b, such as signals from electrodes in response to injected currents that can be converted to impedance information. Additionally, the controller 400 can receive location data 412 such as information regarding the location of the electrode array from the EAM system 70. In some embodiments, the controller 400 can receive an input representative of the anatomical map of the heart, or heart map data 414, which heart map data 414 include the data regarding representations of the geometric anatomical map of the heart and the electro-anatomical map of the heart, such as from the EAM system 70. In some embodiments, the controller 400 receives catheter parameter information 416, such as data specific to the catheter coupled to the controller 400. Further, the controller 400 generates a visualization 420, such as transmits to a graphic display a signal for a visualization of the force and deflection and direction of force and deflection as well as an indication of which splines or electrodes are in contact with tissue. In one embodiment, the visualization is a catheter in contact with tissue and a state of deflection.

In some embodiments, the catheter coupled to the controller includes a memory device (non-transitory memory), such as memory device 103 of catheter 100 in FIG. 1, storing a set of parameters associated with the design and configuration of the catheter. The memory device is coupled to the controller 400 via the plug, such as a serial interface, and the controller is configured to read the parameters to program the controls to be suited for the associated catheter 200. The memory device can store the parameters in various memory segments having lookup tables or other data structures to provide data to be loaded into a memory device 404 in the controller 400 and read by a controller to affect operation. In one embodiment of catheter 200, the memory device is programmed and stored to include strain gauge or other force sensing device parameters such as calibration coefficients to be used in the determination of force and deformation. In one embodiment of manufacturing the catheter 200 with strain gauges, the catheter is subjected to various tests of compression of the expanded electrode assembly 210, such as radial and axial compression, and the amounts of force used to generate respective strain states are recorded. The correlative data of force to strain states can be stored in a lookup table on the memory device or a curve. In some embodiments, the curve includes various stiffness coefficients/calibration curves. In some embodiments, the correlative data is tested and recorded on a catheter-to-catheter basis in manufacturing. During use of the catheter in an ablation or mapping procedure, the data from the memory device on the catheter is loaded into memory 404 of the controller 400 and applied to electrical signals received from the strain gauge.

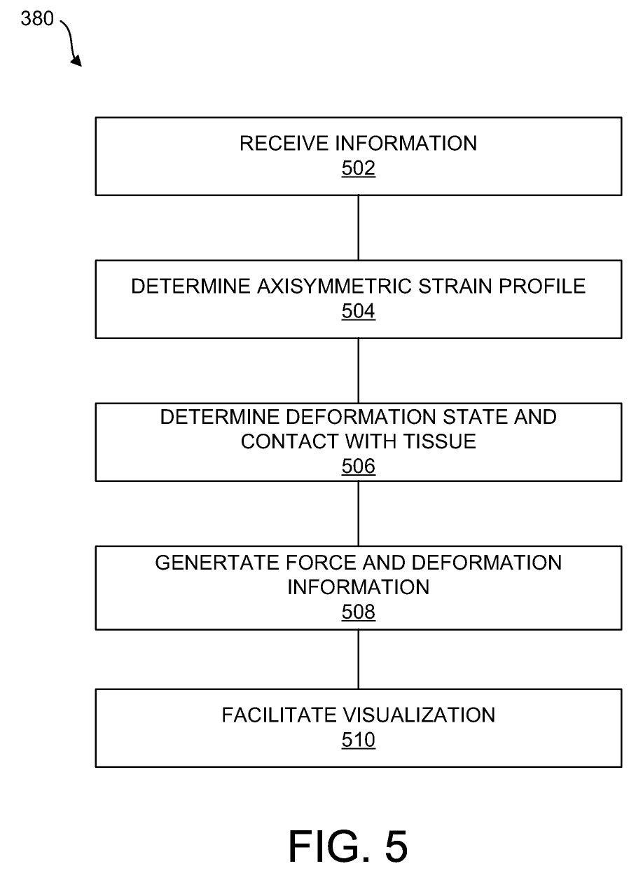

FIG. 5 illustrates a process 500 to configure a controller, such as controller 400. In one embodiment, process 500 can be implemented as set of processor-executable instructions, such as instructions 406, stored in a non-transitory memory, such as memory 404 to be executed by a processor 402 to configure controller 400.

The controller receives information from the catheter at 502. In embodiments, the controller 400 loads correlative strain to force data from the catheter memory device into the memory device 404 of the controller 400 for use with the program 406. In embodiments, the controller 400 can load data from the catheter memory device at initialization. The controller 400 also receives sensor strain signals from the strain gauges and impedance information from the electrodes during operation of the catheter. In one embodiment, the controller 400 causes the electrode assembly 210 to inject currents to current carrying pair of electrodes and then determine voltages from sensing pairs of electrodes as part of receiving impedance data. The controller 400 can cause the activation of the electrodes, and in some embodiments, the controller 400 cycles through several sets of selected sensing pairs of electrodes during an injection of current, such as selected sensing pairs of electrodes are multiplexed in time or frequency, to determine several voltage measurements. In one embodiment, the selected sensing pairs of electrodes are multiplexed in time or frequency while the electrode array is at a relatively stationary location with respect to the heart. In some embodiments, the impedance determinations at are performed either concurrently or sequentially with other functions of the electrode array, such as ablation or other data collection in a known manner. From the voltage measurement of a sensing pair of electrodes based on the injected current from a current carrying pair of electrodes, the controller 400 can determine local impedance as a ratio of the measured voltage to the injected current.

The controller processes the sensor strain signals to determine an axisymmetric strain profile of the electrode array at 504. Each sensor strain signal is representative of an amount of strain applied to the splines at the region of the respective sensor. The sensor strain signals together are applied to create an indication of the regions of the electrode array under strain. As the correlative strain to force data is applied to this information, the controller determines the amount of force placed on the electrode array.

The controller processes the impedance data to determine the deformation state of the electrode array as well as whether which splines are in contact with tissue at 506. The impedance data, and variations in the impedance data can provide an indication of the position of the electrodes in the sensing pair, and thus indicate whether a spline has been deformed from its expanded state. The controller can determine axial and radial compression on the electrode array. The ratio of the measured voltage to the injected current provides an indication of the local impedance, such as the impedance of the medium, through which the current traveled. Based on the impedance measurement, the controller determines if the spline and electrodes are in contact with the tissue or, for example, in the blood pool.

The determinations are combined to generate force and deformation information at 508. The correlative data loaded in memory is applied to the axisymmetric strain profile determined from the sensor strain signal and is combined with determined the axial and radial compression on the electrode array and with the determined splines and electrodes and in contact with tissue to determine forces applied to the electrode array and the direction of the forces. In view of the forces and direction of forces and the state of deformation of the electrode array from the expanded configuration, the controller can determine force and deformation information.

In some embodiments, the controller can facilitate a visualization of a representation of the electrode array based on the force and deformation information to indicate which splines and electrodes are in contact with the tissue and the state of the electrode array under deformation at 510. In one embodiment, a graphical indication of the shape of the electrode array is produced in the visualization. In some embodiments, the visualization can include widgets that indicate an amount of force and direction of force applied to the splines and electrodes. Further, the visualization can highlight whether the contact between the tissue and the splines is sufficient for ablation. The controller receives other information regarding the heart, such as anatomical map data 414 and location data 412 to indicate graphically the shape of the electrode array and spline contact with respect to regions of the heart.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating cardiac tissue through irreversible electroporation, the catheter comprising:
an outer shaft having a proximal end and an opposite distal end;
an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising:
an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end; and
an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines.

2. The catheter of claim 1, further comprising a plurality of spline sensing electrodes located on each spline.

3. The catheter of claim 2, wherein the plurality of spline sensing electrodes are disposed on the outwardly facing flexible circuit.

4. The catheter of claim 1, wherein each spline of the plurality of splines includes an inwardly facing flex circuit branch of the plurality of inwardly facing flex circuit branches.

5. The catheter of claim 4, wherein each spline of the plurality of splines includes a respective outwardly facing flex circuit branch of the plurality of outwardly facing flex circuit branches.

6. The catheter of claim 4, wherein each inwardly facing flex circuit branch includes one strain gauge.

7. The catheter of claim 1, wherein each spline of a subset of the plurality of splines includes an inwardly facing flex circuit branch of the plurality of inwardly facing flex circuit branches.

8. The catheter of claim 7, wherein the plurality of splines includes six splines and the subset of the plurality of splines includes three splines.

9. The catheter of claim 1, further comprising a hub sensing electrode centrally located on the central hub portion of the electrode assembly and a shaft electrode on the outer shaft proximal to the distal end of the outer shaft.

10. The catheter of claim 1, further comprising a post electrode extending distal to the distal end of the outer shaft.

11. A system for ablating cardiac tissue through irreversible electroporation, the system comprising:
a catheter for ablating cardiac tissue through irreversible electroporation, the catheter comprising:
an outer shaft having a proximal end and an opposite distal end;
an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising:
an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end; and
an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines;
a graphical display; and
a controller coupled to the graphical display and the catheter, the controller configured to:
determine an impedance from a four-terminal configuration, wherein a current is injected to a current carrying pair of the plurality of electrodes and a volage is measured across a sensing pair of electrodes comprising a plurality of sensing electrodes;
determine a force applied to the plurality of splines from sensor strain signals received from the inwardly facing strain gauge; and
facilitate a visualization of a representation of the electrode assembly indicating which of the plurality of splines and electrodes are in contact with tissue and the state of the electrode assembly under deformation.

12. The system of claim 11, wherein the controller is configured to cycle through several sets of selected sensing pairs of electrodes during an injection of current.

13. The system of claim 11, wherein the controller is configured to receive correlative strain to force data from the catheter.

14. The system of claim 13, wherein controller is configured to determine an axisymmetric strain profile of the electrode assembly from the sensor strain signals.

15. The system of claim 14, wherein the controller is configured to apply the correlative strain to force data loaded to the axisymmetric strain profile and with a determined axial and radial compression on the electrode assembly to determine forces applied to the electrode assembly and a direction of the forces.

16. The system of claim 11, further comprising a plurality of spline sensing electrodes located on each spline, wherein the plurality of spline sensing electrodes are disposed on the outwardly facing flexible circuit.

17. The system of claim 11, wherein each spline of the plurality of splines includes an inwardly facing flex circuit branch of the plurality of inwardly facing flex circuit branches, wherein each inwardly facing flex circuit branch includes one strain gauge.

18. A method for ablating cardiac tissue through irreversible electroporation including a catheter having an electrode assembly extending distally from the distal end of the outer shaft, the electrode assembly defining a distally located central hub portion and a plurality of splines each including a distal end portion extending from the central hub portion, a proximal end portion attached to and constrained by the outer shaft, and an intermediate portion disposed between the distal end portion and the proximal end portion, each of the plurality of splines also including an outwardly facing portion and an inwardly facing portion, the electrode assembly comprising an outwardly facing flexible circuit disposed on the outwardly facing portions and having a flex circuit hub and a plurality of outwardly facing flex circuit branches extending proximally from the flex circuit hub, the outwardly facing flexible circuit further including an outwardly facing ablation electrode including an ablation electrode hub portion located on the flex circuit hub and a plurality of outwardly facing radial segments integrally formed with the ablation electrode hub portion, each of the outwardly facing radial segments extending proximally along a portion of a respective one of the outwardly facing flex circuit branches and terminating in a proximal end, and an inwardly facing flexible circuit disposed on the inwardly facing portions of the plurality of splines and having a plurality of inwardly facing flex circuit branches, the inwardly facing flexible circuit including an inwardly facing strain gauge disposed on the inwardly facing and intermediate portions of the plurality of splines, the method comprising:
determining an impedance from a four-terminal configuration, wherein a current is injected to a current carrying pair of the plurality of electrodes and a volage is measured across a sensing pair of electrodes comprising a plurality of sensing electrodes;
determining a force applied to the plurality of splines from sensor strain signals received from the inwardly facing strain gauge; and
facilitating a visualization of a representation of the electrode assembly indicating which of the plurality of splines and electrodes are in contact with tissue and the state of the electrode assembly under deformation.

19. The method of claim 18, further comprising receiving correlative strain to force data from the catheter and determining an axisymmetric strain profile of the electrode assembly from the sensor strain signals.

20. The method of claim 19, further comprising applying the correlative strain to force data loaded to the axisymmetric strain profile and with a determined axial and radial compression on the electrode assembly to determine forces applied to the electrode assembly and a direction of the forces.
